(19) United States
(12) Patent Application Publication
(10) Pub. No.: US 2005/0059856 A1
(43) Pub. Date: Mar. 17, 2005

(54) Kissing game for two persons

(76) Inventor: Mara Fox, Marina Del Rey, CA (US)

Correspondence Address: DON E. ERICKSON;LAW OFFICE, 7668 EL CAMINO REAL STE. 104 #627, LA COSTA, CA, 92009, US

(21) Appl. No.: 10/644,930

(22) Filed: Aug. 21, 2003

(51) Int. Cl. A61F 5/00

(52) U.S. Cl. 600/38

(57) ABSTRACT

The present invention describes a novelty method and apparatus whereby two parties create taste sensations by one or both persons placing a soluble, flavored substance on the tongue, and then deep kissing, and using their respective tongues to cause the substance(s) to be placed, or commingled, on the tongues of both individuals, thereby producing taste sensations for both persons.

### BACKGROUND OF THE INVENTION

1. Field of the Invention.

The present invention relates to a kissing game for two persons, and more particularly, to a game in which two persons can produce varied taste sensations through kissing.

2. Background of the Invention.

Kissing, the act of osculation between two persons, is a form of greeting or salutation that has a long history in Western Civilization, dating back to the ancient Greeks and Romans, and has been referenced in the Old Testament. Under Western influence, kissing in public gradually became more common in Japan and China in the late 20th century. Kissing as an inducement to sexual readiness is unique to humans, and deep kissing, in which the tongue is used to explore the other person's mouth, usually occurs in an erotic context, either to express affection or as a means of sexual arousal.

Tasting occurs in the mouth, chiefly on the tongue through the taste buds. The taste buds are stimulated by four fundamental taste sensations: sweet, salty, sour, and bitter. Examples of substances that induce the four basic tastes are sugar (sweet), salt (salty), lemon juice (sour), and quinine (bitter). Substances can be tasted only when they are in water solutions, and if a substance is not in solution when taken into the mouth, it must be dissolved in saliva before it can be detected by the taste buds. The taste buds most sensitive to salty flavors are dispersed along the sides and front of the tongue. Taste buds sensitive to sweet flavors are concentrated on the tip of the tongue. Bitter flavors are detected at the rear of the tongue and sour flavors on the sides.

It is well known that there are a variety of extracts available on the market that provide various tastes, common of which is vanilla. However, these extracts are concentrated, and are not suitable for the uses contemplated in the invention.

The present invention is a novelty device whereby two persons can exchange taste sensations through deep kissing. The only known prior art is the typical “breath freshener,” the ingredients of which are not selected on the basis of the flavor to produce a certain taste to the user, or demonstrate a particular mood, but whose ingredients are selected solely for the basis of eliminating breath odors. Such breath fresheners teach away from the intent of the present invention in the endeavor of removing tastes, rather than imparting them.

### SUMMARY OF THE INVENTION

The present invention describes a novelty method and apparatus whereby two parties create taste sensations by one or both persons placing a soluble, identifiable substance on one or both of their respective tongues, and then deep kissing, and using their respective tongues to cause the substance(s) to be placed, or commingled, on the tongues of both individuals, thereby producing taste sensations for both persons. The method and apparatus includes providing substances of varying tastes, and identifying various moods that may be created by the commingling of such tastes, along with instructions on their use.

### DETAILED DESCRIPTION OF THE INVENTION

The present invention is a novelty kit for providing manufactured consumables to facilitate two individuals in providing complementary taste sensations. Each kit contains (1) one or a plurality of consumables substances having various taste sensations which, when placed on the tongue of one individual, may be transmitted to the tongue of second individual, and (2) instructions for the use of the consumable substance. It is well known that certain consumable substances have tastes that are popular with many individuals, having attributes (apart from texture and temperature) that are produced by the senses of smell, taste, and touch that are perceived within the mouth. These sensations help to identify substances and are sources of enjoyment when eating and drinking. Exemplars of these are various fruits, alcohols, foods, and nuts. Many variations of candies and ice cream flavors are produced for human consumption based on the wide popularity of the tastes created. For example, chocolate is recognized worldwide as a delicious and pleasant consumable substance, and is produced in a number of forms as candies, ice cream, and as an additive to flavor various drinks, such as milk. It is contemplated that both parties may place consumable substances on their tongues so that the tastes may be co-mingled on the tongues of each person through the act of deep kissing.

In the preferred embodiment, selected flavors/essences of substances are dissolved in a carrier, such as spring water, and placed in a vials, or vessels, that resemble typical ‘breath freshener’ bottles, that are reasonably “tamper free,” and which enables the user to control the amount and location of solute placed on the tongue. Alternatively, vials, or vessels, may be employed wherein the top of the vial has incorporated therein a dropper, as well known by those of ordinary skill in the art, and commonly known as an eye-dropper. Care need be exercised with this second manner to ensure that the content of the vial cannot be tampered with. It is also contemplated that the substances of the kit of the invention could be provided in single-use capsules wherein one dosage of the selected substance is contained in a sealed, plastic container, which capsule is then disposed of upon release of the substance.

As noted above there are a wide variety of substances having unique and identifiable tastes that may be provided. Solutes of these substances may be produced from over-the-counter food products and extracts. The solute may be water, juice, and/or alcohol. A user would be provided a kit containing at least one substance or solute, with instructions on how the substance or solute would be employed by one or both of the individuals. Individual bottles of varying taste sensations may also be supplied, providing for a variety of taste combinations. The solutes are not restricted to having sweet tastes. Solutes containing the juices of lemon, or lime, may also be included, providing the users to experiment in the combination of sweet and sour tastes.

It is known to those that practice various folklore that different tastes, flavors and scents produce different feelings with a consumer, and these feelings, or sensations, may vary between individuals. For example, just as flowers have meaning, (red roses signify love; carnations—devotion; marigolds—sorrow); and that stones have meaning, (diamond—fidelity; malachite—wealth; turquoise—friendship), so too, do tastes and flavors. While the centuries old traditions signifying the meanings of stones, flowers, and even scents have been much capitalized upon over the past several decades, products utilizing the meanings of flavors have not yet been tapped.

The basis for the instant invention is that a choice of taste or flavor will signify a mood that a person is in, or may want to create. Thus, in a preferred embodiment of the invention, a kit of the invention may initially include a variety of packages having six tastes/flavors each. The consumer may select which of a number of combinations he or she may select. Ingredients may include, but are not limited to, a combination of the highest premium quality all-natural extracts and essences available on the market, of a type and quality usually only available to/or sought by professional chefs. These extracts are mixed with liquid cane sugar blends using product from a small label manufacturer prided on the exceptional quality of its products. When needed, according to selected factors of taste and opinion, they may be then mildly diluted with the purest and most delicious mineral spring water. The result is a drop of heaven in a bottle.

In this exemplary embodiment, while the bottles may contain the flavor of chocolate or vanilla, a fruit, a mint, or a nut, etc., they are not labeled as such. They are labeled by their “meaning” only. Thus, a consumer may choose a flavor labeled to reflect a particular mood. For example, one party may choose a bottle marked “#3—FUN”. His partner may choose a bottle named “#7—PLAYFUL”. They each place a drop of liquid on their respective tongues from vessels that resemble typical ‘breath freshener’ bottles. They then first touch tongues, then kiss deeply. One can then determine it the persons' moods match, or if the flavors combine well. It is known that certain flavors combine well, such as chocolate and cherry. It is known that “fun” is chocolate, “playful” is “cherry”, and those two combine very well indeed. Not so in all cases though. Based on the preferences of the individual, possibly, with banana and mint, the persons may never again kiss. And thus, the basis for the novelty game, wherein one individual may place the solute on their tongue, and upon deep kissing, the sensory system of the other individual may be challenged to identify the solute and/or common tastes. Additionally, a solute may be placed on the tongues of both individuals, and the solutes mixed during deep kissing, providing a combined sensatory sensation for establishing moods and testing compatibility between the users.

The instructions may include a pamphlet with a ‘decoder’ list detailing the actual flavors that the mood names signify. There is also space that allows for users to write down their favorite combinations, and their favorite kissers. From this, the users may determine, are our moods compatible at this time? Are we compatible at all?

While the present description contains much specificity, this should not be construed as limitations on the scope of the invention, but rather as exemplifications of one/some preferred embodiment/s thereof. A kit may contain a single flavor, or a plurality of flavors, instructions may require a flavor be placed on the tongue of one person, or on the tongues of both. Flavors may be identified by their source, or by effect. The solute may be an alcohol, or substance other than water in which the substance will dissolve. Accordingly, the scope of the invention should not be determined by the specific embodiments illustrated herein. The full scope of the invention is further illustrated by the claims appended hereto.

1. A novelty kit for producing an oral sensation during deep kissing, the novelty kit comprising:
a) a first substance to be placed on the tongue of a first person; and
b) instructions for use of the first substance with a second person.

2. The novelty kit of claim 1 wherein the first substance has an identifiable taste.

3. The novelty kit of claim 1 wherein the first substance is in the form of a solute containing an extract of the first substance.

4. The novelty kit of claim 3 where the solute is water based.

5. The novelty kit of claim 2 additionally comprising a second substance, and wherein the instructions include directions for the use of the first substance and the second substance by the first person and the second person.

6. The novelty kit of claim 5 wherein the second substance has a different identifiable taste than the taste of the first substance.

7. The novelty kit of claim 5 wherein the second substance is in the form of a solute containing an extract of the second substance.

8. The novelty kit of claim 7 where the solute is water based.

9. The novelty kit of claim 1 wherein the instructions direct the first person on placement on the tongue of the first substance prior to the first and second person engaging in deep kissing.

10. The novelty kit of claim 5 wherein the instructions direct the first and second persons on placement on their respective tongues of the first and second substances prior to the first and second person engaging in deep kissing.

11. The novelty kit of claim 5 wherein the instructions direct the first and second persons on the selection of tastes of the first and second substances.

12. The novelty kit of claim 5 wherein the instructions identify moods created by the selected tastes and wherein the instructions direct the first and second persons on the selection of the first and second substances to create a selected mood.

13. A method for producing an oral sensation during deep kissing, the method comprising:
a) placing a first substance having an identifiable taste on the tongue of a first person; and
b) instructing the first person on use of the first substance with a second person.

14. The method of claim 13 wherein the first substance is in the form of a solute containing an extract of the first substance.

15. The method of claim 14 wherein the solute is water based.

16. The method of claim 13 additionally including the steps of placing a second substance having an identifiable taste on the tongue of a second person, and instructing the first and second persons on the use of the first and second substances.

17. The method of claim 16 wherein the second substance is in the form of a solute containing an extract of the second substance.

18. The method of claim 17 wherein the solute is water based.

19. The method of claim 13 wherein the step of instructing includes directing the first person on placement on the tongue of the first substance prior to the first and second person engaging in deep kissing.

20. The method of claim 16 wherein the step of instructing includes directing the first and second persons on placement on their respective tongues of the first and second substances prior to the first and second person engaging in deep kissing.

21. The method of claim 16 wherein the step of instructing includes directing the first and second persons on the selection of tastes of the first and second substances.

22. The method of claim 16 wherein the step of instructing includes identifying moods created by the selected tastes and directing the first and second persons on the selection of the first and second substances to create a selected mood.
